(19) United States
(12) Patent Application Publication
(10) Pub. No.: US 2014/0088716 A1
(43) Pub. Date: Mar. 27, 2014

(54) VARIABLE DENSITY IMPLANT AND METHOD

(71) Applicant: Zimmer, Inc., Warsaw, IN (US)

(72) Inventors: Ray Zubok, Midland Park, NJ (US); Mehul Dharia, Albuquerque, NM (US); Joseph R. Vargas, Garnerville, NY (US); Steven Seelman, Montclair, NJ (US); Timothy A. Hoeman, Morris Plains, NJ (US)

(73) Assignee: Zimmer, Inc., Warsaw, IN (US)

(21) Appl. No.: 14/032,705

(22) Filed: Sep. 20, 2013

Related U.S. Application Data
(60) Provisional application No. 61/703,946, filed on Sep. 21, 2012.

(51) Int. Cl. A61F 2/30; A61F 2/28

(52) U.S. Cl. CPC A61F 2/30; A61F 2/28; A61F 2/3094
USPC 623/18.11; 623/16.11; 427/2.26

(57) ABSTRACT

An implant can comprise a porous region including a plurality of interconnecting interstitial cells configured to receive bone or biological tissue ingrowth. The porous region can be formed of a first portion, including a first plurality of the interconnecting interstitial cells and having a first density, and a second portion, including a second plurality of the interconnecting interstitial cells and having a second density different than the first density. The plurality of interconnecting interstitial cells can form a framework onto which a material can be disposed in different amounts to provide the first and second densities of the first and second portions, respectively. The second density can be selected and configured to provide greater bone or biological tissue ingrowth than the first density. The first and second density can be selected and configured to substantially match an anisotropic property of a body component the implant is intended to replace or augment.

### CLAIM OF PRIORITY

This application claims the benefit of U.S. Provisional Patent Application Ser. No. 61/703,946, filed on Sep. 21, 2012, the benefit of priority of which is claimed hereby, and which is incorporated by reference herein in its entirety.

### BACKGROUND

Prosthetic devices, such as prosthetic implants, can replace or augment body components or portions of body components that cannot be regenerated or are no longer functioning properly. Examples of prosthetic implants include heart valves, pacemakers, spinal implants, dental implants, breast implants, collagen for soft tissue augmentation, and orthopedic devices, such as artificial knee, hip, and ankle joints.

Some prosthetic implants can include a porous scaffold material. Porous scaffold materials can be used to provide structural support to a patient's tissue, such as bone tissue. Porous scaffold materials can also be used to provide an attachment structure for a patient's tissue to couple, attach, or bond, such as via ingrowth.

U.S. Pat. No. 5,282,861 is directed toward open cell tantalum structures for prosthetic cancellous bone implants and cell and tissue receptors.

### SUMMARY

The present inventors have recognized, among other things, that an implant's strength can be directly related to an amount of material present at stress locations, with more material resulting in more strength. However, more material can result in an unnecessarily heavy or cost prohibitive implant.

One way to improve an implant's strength without adding prohibitive weight can be to increase an amount of material present at one or more stressed locations of the implant and reduce the amount of material present at one or more unstressed or lower stress locations of the implant.

To better illustrate the variable density implant and related methods disclosed herein, a non-limiting list of examples is provided here:

In Example 1, an apparatus comprises an implant including a porous region including a plurality of interconnecting interstitial cells configured to receive bone or biological tissue ingrowth. The porous region can include or be defined by a first portion, including a first plurality of the interconnecting interstitial cells and having a first density, and a second portion, including a second plurality of the interconnecting interstitial cells and having a second density different than the first density.

In Example 2, the apparatus of Example 1 is optionally configured such that the first portion includes a structured region having a larger topology variation than any topology variation associated with the second portion, the second portion being located outside of the structured region.

In Example 3, the apparatus of Example 2 is optionally configured such that the structured region includes a threaded region having one or more threads.

In Example 4, the apparatus of any one or any combination of Examples 2 and 3 is optionally configured such that the first density associated with the first portion is greater than the second density associated with the second portion.

In Example 5, the apparatus of any one or any combination of Examples 1-4 is optionally configured such that the first density associated with the first portion is greater than the second density associated with the second portion by an amount corresponding to a chemical vapor deposition (CVD) process variation.

In example 6, the apparatus of any one or any combination of Examples 1-5 is optionally configured such that the first density associated with the first portion is greater than the second density associated with the second portion by at least 5 percent.

In example 7, the apparatus of any one or any combination of Examples 1-6 is optionally configured to include ligaments and one or more pores, each pore having a smaller size than the surrounding cell.

In example 8, the apparatus of Example 7 is optionally configured such that the first portion includes one or more pores having a first central tendency of pore diameter. The second portion can include one or more pores having a second central tendency of pore diameter, the second central tendency of pore diameter being larger than the first central tendency of pore diameter.

In Example 9, the apparatus of any one or any combination of Examples 1-8 is optionally configured to include a framework and a material that is deposited on the framework in one or more different amounts to provide the first and second densities of the first and second portions, respectively.

In Example 10, the apparatus of any one or any combination of Examples 1-9 is optionally configured such that the first density is configured to provide a stress tolerance that is greater than a stress tolerance provided by the second density.

In Example 11, the apparatus of any one or any combination of Examples 1-10 is optionally configured such that the second portion is configured to provide greater bone or biological tissue ingrowth than the first portion.

In Example 12, the apparatus of any one or any combination of Examples 1-11 is optionally configured such that the first density or the second density is configured to substantially match a predetermined anisotropic property

In Example 13, a method comprises vapor depositing a material on a porous framework including a plurality of interconnecting interstitial cells configured to receive bone or other biological tissue ingrowth and controlling a rate of the vapor deposition of the material on one or more portions of the framework, including varying a density of the material across the framework to provide a first porous portion, including a first plurality of the interconnecting interstitial cells and having a first density, and a second porous portion, including a second plurality of the interconnecting interstitial cells and having a second density less than the first density.

In Example 14, the method of Example 13 is optionally configured such that varying porosity density includes forming a first central tendency diameter of a plurality of pores included in the first porous portion and forming a second central tendency diameter, which is greater than the first central tendency diameter, of a plurality of pores included in the second porous portion.

In Example 15, the method of any one or any combination of Examples 13 and 14 optionally further comprises providing the first porous portion at a structured region, the structured region having a larger topology variation than any topology variation associated with the second porous portion.

In Example 16, the method of any one or any combination of Examples 13-15 optionally further comprises positioning the framework within a deposition reactor according to a temperature distribution of the deposition reactor. In Example 17, the method of any one of or any combination of Examples 13-16 optionally further comprises positioning the framework within a deposition reactor according to a predetermined porosity for the first portion and a predetermined porosity for the second portion.

In Example 18, the method of any one or any combination of Examples 13-17 optionally further comprises selecting a porosity for the first portion or the second portion according to a predetermined stress tolerance distribution of the implant.

In Example 19, the method of any one or any combination of Examples 13-18 optionally further comprises directing vapor deposition flow using a shield.

In Example 20, the method of any one or any combination of Examples 13-19 optionally further comprises altering the rate or a material concentration of the vapor deposition.

In Example 21, the method of any one or any combination of Examples 13-20 optionally further comprises providing the first and second porous portions on opposing sides of the implant.

In Example 22, an apparatus comprises an implant including a porous region including a plurality of interconnecting interstitial cells configured to receive bone or biological tissue ingrowth. The porous region can comprise a first portion that includes a first plurality of the interconnecting interstitial cells and has a first density, and a second portion that includes a second plurality of the interconnecting interstitial cells specified to have a second density of the cells, the second portion is configured to provide a lower stress tolerance and greater bone or biological tissue ingrowth than the first portion.

In Example 23, the apparatus of Example 22 optionally configured such that the first portion is positioned at a predetermined stress concentration region realized during implant insertion, use, or removal.

In Example 24, the implant or method of any one or any combination of Examples 1-23 is optionally configured such that all elements or options recited are available to use or select from.

These and other examples and features of the present prosthetic implants and methods will be set forth in part in the following Detailed Description. This Summary is intended to provide non-limiting examples of the present subject matter—it is not intended to provide an exclusive or exhaustive explanation. The Detailed Description below is included to provide further information about the present prosthetic implants and methods.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a perspective view of a variable density implant in accordance with at least one example;

FIG. 2 illustrates an enlarged fragmental view of a porous portion of the variable density implant of FIG. 1 in accordance with at least one example;

FIG. 3 illustrates a deposition reactor in accordance with at least one example; and

FIG. 4 illustrates method steps for varying the density of implant portions in accordance with at least one example.

### DETAILED DESCRIPTION

The present disclosure relates generally to a variable density implant and related method of manufacture. Generally, implants can be manufactured such that the implant as a whole can withstand a stress experienced by an isolated portion of the implant. For example, an implant can be designed and manufactured such that the entire implant can withstand an acute stress event realized by an isolated portion of the implant. Such design and manufacture typically results in an increased amount of material throughout the implant, such that the implant is unnecessarily heavy, bulky, or costly. According to the present disclosure, an implant can include one or more portions of varying density. Benefits of such a design can include a reduced weight, an increased implant stress tolerance, or a more customizable implant as compared to current implants. Further, a variable density implant can provide the benefit of increased safety to a patient during everyday use due to the individualized design of each component. For example, the density can be increased in implant areas where higher stress is likely, or the porosity of an implant area can be increased in areas of likely lesser stress, such that bone in-growth can be encouraged. Additionally, the variable density implant can substantially match or replicate anisotropic properties of the bone or biological structure it is intended to replace or fill, such as when the implant is used as a bone void filler. For example, a portion of the implant replacing cortical bone can be designed with increased material density (e.g., decreased porosity) and a portion of the implant representing cancellous bone can be designed with reduced material density (e.g., increased porosity).

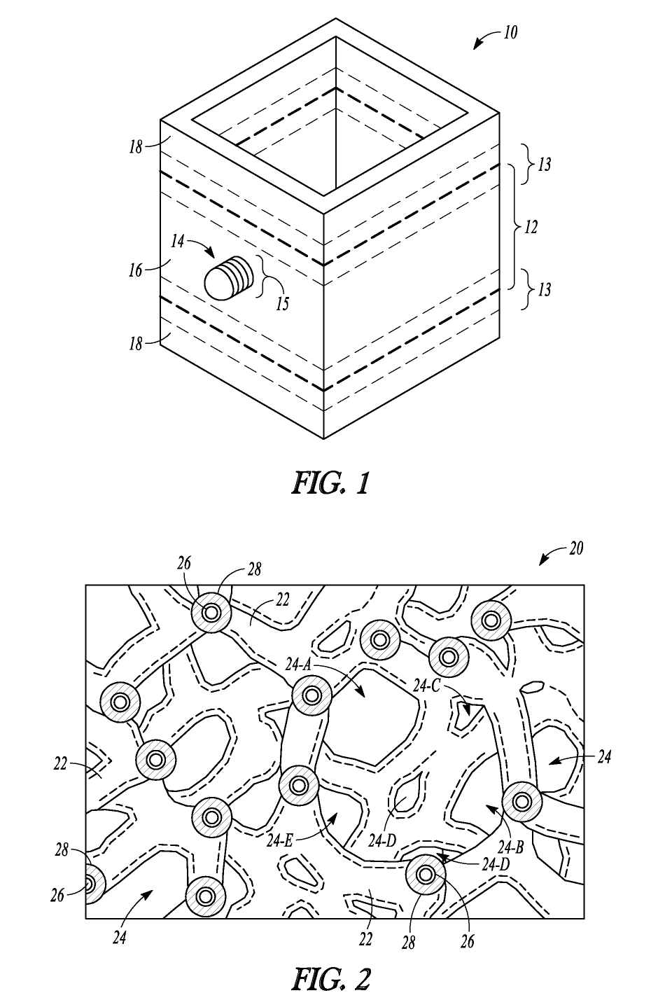

As shown in FIG. 1, a variable density implant 10 can include a porous region 12. The implant 10 can include any medical device suitable to replace a missing biological structure, support a damaged biological structure, or enhance an existing biological structure. Suitable example implants include, but are not limited to, an intramedullary nail, a bone plate, the components that constitute a reverse shoulder prosthesis, a hip prosthesis, a knee implant, an ankle implant, spinal implants, dental implants, and the like. An implant material can include any biocompatible material suitable for implantation in a patient for any given length of time. Suitable biocompatible materials can include, for example, a metallic material such as at least one of a variety of stainless steel composites, titanium, chromium-cobalt, tantalum, or the like, or a non-metallic biocompatible material such as a biocompatible polymeric or other plastic material including polyamide, polyphenylsulfone, polyethersulfone, polysulfone, polyketone, polyarylamide, polyether ether ketone (PEEK), polycarbonate, polystyrene, acrylonitrile butadiene styrene (ABS), acrylics, polyetherimide, polyimide, polyphenylsulfone, polymethoylmethacrylate, fiber filled variations of these polymers, amorphous polymeric material, or various other biocompatible polymers.

The porous region 12 can include a plurality of interconnecting interstitial cells configured such that bone or biological tissue ingrowth can occur within the cells, as described in connection with FIG. 2. Although shown as a discrete and continuous region of the implant 10, the porous region 12 is not so limited. For example, the porous region 12 can include an outer surface of an implant, an interior surface of an implant, or any region of an implant that bone or biological tissue ingrowth is desired. In an example, the porous region 12 is the only region of the implant 10.

The implant 10 can include a non-porous region 18. Although only one non-porous region 18 is shown in FIG. 1, examples are not so limited. For example, the implant 10 can include any number of non-porous regions, such as two, three, four, or any number associated with a type or function of the implant 10. A non-porous region can be made of a suitable biocompatible material, such as at least one of the following: titanium alloy, stainless steel, zirconium, cobalt-chromium molybdenum alloy, ceramic, a polymer, and a composite material.

The porous region 12 can include a first portion 14, including a first plurality of the interconnecting interstitial cells specified to have a first density. A second portion 16 of the porous region 12 can include a second plurality of the interconnecting interstitial cells specified to have a second density. Examples are not limited to only first and second porous portions. For example, the implant 10 can include more than two porous portions 14 and 16, such as three, four, five, or more. The number of porous portions can be dependent on the type or function of the implant 10.

The first density can be different than the second density. The first and second densities can be specified according to a stress distribution of the implant 10. The stress distribution can represent acute stress events, which the implant 10 can experience during implantation, use by a patient, or during extraction. The first and second densities can correlate to strength properties of an implant material, which can withstand such acute stress events. For example, the first density can be specified to exceed the second density, such that the first portion 14 including the first density can withstand a greater stress event than the second portion 16 including the second density. In an example, the first density can be specified to exceed the second density, such that the second portion 16 including the second density can provide greater ingrowth or fixation as compared to the first portion 14 including the first density. Such examples can provide targeted stability or fixation of the implant 10 or targeted material strength of the implant 10.

In an example, the first density can exceed the second density by an amount that corresponds to a chemical vapor deposition (CVD) process variation. A CVD process variation can include any measureable or controllable process variable during a deposition process, such as temperature, vapor concentration, vapor composition, position within a deposition reactor, orientation of a framework within the deposition reactor, vapor flow distribution, or length of time of the deposition process. In varying examples, the first density is specified to exceed the second density by at least 5 percent, at least 10 percent, at least 15 percent, at least 20 percent, at least 25 percent, or at least 30 percent or more. The description in range format is merely for convenience and brevity and should not be constructed as an inflexible limitation on the scope of the present disclosure. Accordingly, the description of a range (e.g., in at least numerical percent format) should be considered to have specifically disclosed all possible subranges as well as individual numerical values within each range.

In an example, the first density of the first porous portion 14 or the second density of the second porous portion 16 can be designed to substantially replicate a predetermined anisotropic property, such as elasticity, density, strength, or stress tolerance. The predetermined anisotropic property can include an anisotropic property of the body component the implant 10 is intended to augment or replace. The term “substantially”, as used herein, refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The implant 10 can include a structured region 15 configured to provide a larger topology variation than any topology variation associated with the porosity of a surrounding porous region 12. In an example, the first portion 14 of the porous region 12 is located within the structured region 15 and the second portion 16 of the porous region 12 is located outside of the structured region 15. The structured region 15 can include any type of topology variations including, but not limited to, one or more threads, protrusions, recesses, orifices, roughened surfaces, or the like. The structured region can include any topology variation designed to serve predetermined function of the implant 10. As show in FIG. 1, the structured region 15 can include a threaded region including one or more threads that are configured to provide the larger-sized topology variation. The threaded region can include external or internal threads.

In an example, the first porous portion 14 can include first pores having a first central tendency of pore diameter and the second porous portion 16 can include second pores having a second central tendency of pore diameter. The first central tendency can be smaller than the second central tendency. Central tendency can include an average pore diameter, a median pore diameter, or some other statistical measure correlating to a commonality of pore diameter within the first or second porous portions 14 and 16, respectively. An implant material or portion including pores having a smaller central tendency of pore diameter can correlate to a greater material density. That is, a smaller central tendency of pore diameter can correlate to a greater amount of material present within a given area. Such an implant material or portion configuration can provide a greater stress threshold and find utility at locations of the implant where a greater amount of stress is experienced or likely to be experienced. A larger central tendency of pore diameter can correlate to a lower material density and a greater potential for ingrowth of bone or biological tissue within the material. Such an implant material or portion configuration can provide better fixation or stability of the implant 10 following implantation in a patient.

In an example, the first density of the first porous portion 14 can be specified to provide a desired stress tolerance for a specified use of the implant 10. The second density of the second porous portion 16 can be specified to provide a lesser stress tolerance than the first density and allow for more bone or biological tissue ingrowth than the first porous portion 14. The specified use can include implantation of the implant 10, use of implant, or extraction of the implant, for example.

The implant 10 can include one or more transition regions 13 between the boundaries of the porous 12 and non-porous 18 regions. In an example, the one or more transition regions 13 do not include defined boundaries, such that the implant 10 can include multiple gradual transitions to different densities or porosities. The one or more transition regions 13 can be configured according to a rate of change of a process variation, such as vapor deposition rate, temperature, implant orientation, implant position within a reactor, vapor flow distribution, and vapor concentration. Adjusting or varying a process variation can affect a rate at which the one or more transition regions 13 change from porous to non-porous or vice-versa. In an example, the one or more transition regions 13 can be configured to be less porous than the porous region 12, but more porous than the non-porous region 18.

FIG. 2 illustrates an enlarged fragmental view of a porous portion 20 of a variable density implant 10, such as the implant shown in FIG. 1. Examples of porous material can include a material produced using Trabecular Metal™ technology generally available from Zimmer, Inc., of Warsaw, Ind. Trabecular Metal™ is a trademark of Zimmer Technology, Inc. Such a material can be formed from a reticulated vitreous carbon foam substrate which is infiltrated and coated with a biocompatible metal, such as tantalum, etc., by a chemical vapor deposition (“CVD”) process in the manner disclosed in detail in U.S. Pat. No. 5,282,861, the disclosure of which is incorporated herein by reference. Other metals such as niobium or alloys of tantalum and niobium with one another or with other metals can also be used.

Generally, as shown in FIG. 2, the porous portion 20 can include a plurality of ligaments 22 defining pores 24 therebetween, with each ligament 22 generally including a carbon core 26 covered by a thin film of metal 28 such as tantalum, for example. Other core or framework materials as well as other materials for coating or covering such cores are discussed elsewhere herein. The pores 24 between the ligaments 22 can form a matrix of continuous channels having few or no dead ends, such that growth of cancellous bone through the porous portion 20 is substantially uninhibited. The porous portion 20 can include up to 75%-85% or more void space therein. Thus, the porous portion 20 can be a lightweight, strong porous structure that is substantially uniform and consistent in composition, and closely resembles the structure of natural cancellous bone. The porous portion 20 can provide a matrix into which cancellous bone may grow to anchor an implant 10 (FIG. 1) into the surrounding bone of a patient, which increases the stability of the implantation. A rough exterior surface of the implant 10 can provide a relatively high friction coefficient with adjacent bone to further increase initial stability.

In an example, the porous portion 20 can include a plurality of interstitial cells interconnected by ligaments 22 and defining one or more pores 24. Each pore 24 can have a smaller size than the surrounding cells. For example, as shown in FIG. 2, an interstitial cell can include the pores 24-A through 24-E.

In an example, the porous portion 20 can be included in the porous region 12, as shown in FIG. 1. The porous portion 20 can include a framework defining a desired porosity characteristic and a material deposited on the framework in different amounts to provide the first and second portions, e.g., 14 and 16, respectively, of FIG. 1, including the first and second densities. The material can be deposited on the framework differently according to a process variation, for example. The porous portion 20 can be made to include a variety of densities in order to selectively tailor an implant for a particular application or stress tolerance. In particular, a porous tantalum or other porous metal implant, for example, can be fabricated to virtually any desired porosity and pore size, and can thus be matched with the surrounding natural bone in order to provide an improved matrix for bone ingrowth and mineralization. A gradation of pore size on a single implant can be designed such that pores are larger on a first side to match cancellous bone and smaller on a second side to match cortical bone, or even to receive soft tissue ingrowth.

A porous tantalum implant, for example, can be made denser with fewer pores in areas of high mechanical stress or, instead of smaller pores in the tantalum, the implant can be made denser by filling all or some of the pores with a solid material. The solid material can provide additional initial mechanical strength and stability to the porous implant structure and can be selected from a non-resorbable polymer or a resorbable polymer, for example. Examples of non-resorbable polymers for infiltration of the porous structure can include a polyaryl ether ketone (PAEK) such as polyether ketone ketone (PEKK), polyether ether ketone (PEEK), polyether ketone ether ketone ketone (PEKEKK), polymethylacrylate (PMMA), polyetherimide, polysulfone, and polyphenolsulfone. Examples of resorbable polymers can include polylactic co-glycolic acid (PLGA), polylactic acid (PLA), polyglycolic acid (PGA), polyhydroxybutyrate (PHB), and polyhydroxyvalerate (PHV), and copolymers thereof, polycaprolactone, polyanhydrides, and polyorthoesters.

By providing additional initial mechanical strength and stability with a resorbable filler material, a titanium reinforcing implant core, for example, can potentially be removed from the implant. For example, the resorbable material can resorb as the bone grows in and replaces it, which maintains the strength and stability of the implant.

Instead of, or in addition to, porous tantalum or porous metal, an implant can be made of a first material that promotes bone growth or strengthens the implant instead of porous tantalum, such as organic bone graft (e.g., autograft, allograft, xenograft), resorbable polymer (e.g., polylactic co-glycolic acid (PLGA), polylactic acid (PLA), polyglycolic acid (PGA), polyhydroxybutyrate (PHB), and polyhydroxyvalerate (PHV)), non-resorbable polymer, synthetic bone material such as hydroxyapatite (HA), or collagen. An implant of such material can be initially formed and then press-fit into a thread of a different material, as described above, or the thread may be formed on the implant in other ways.

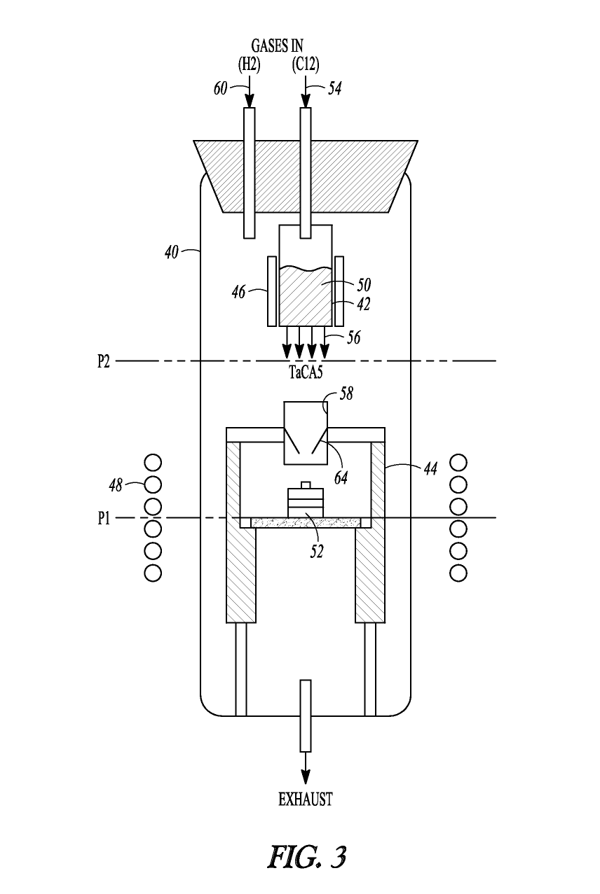

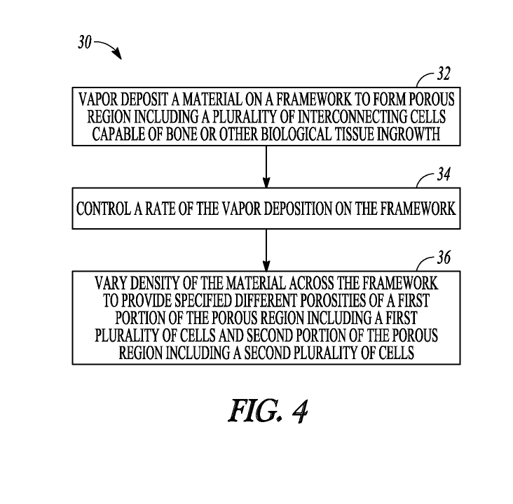

FIG. 3 illustrates an apparatus for depositing a material, such as tantalum metal 50 or other metal or metal alloy, on a framework 52 of an implant 10 (FIG. 1) including a plurality of interconnecting interstitial cells. A deposition reactor 40 can enclose a chlorination chamber 42 or a hot wall furnace 44. A resistance heater 46 can surround the chlorination chamber 42. An induction heating coil 48 can surround the deposition reactor 40 to heat the hot wall furnace 44.

The tantalum metal 50 can be located within the chlorination chamber 42 and a carbon foam substrate framework 52 can be positioned within the hot wall furnace. Chlorine gas, as shown by arrow 54, can be injected into the chlorination chamber 42 to react with the tantalum metal 50 to form tantalum chloride, as shown by arrow 56. The tantalum chloride can mix with hydrogen injected into the reactor 40, as shown by arrow 60, and then pass through an opening 58 in the hot wall furnace 44. In an example, the opening 58 can include a shield 64. The shield 64 can aid in directing a vapor deposition flow mixture including tantalum chloride and hydrogen to the framework 52. The vapor deposition flow mixture can be heated within the hot wall furnace 44 at a temperature of approximately 1100° C., for example, to produce the following surface reaction: Ta.Cl.sub.5+5/2 H.sub.2 Ta+5 HCl. The surface reaction can deposit tantalum on the framework 52 and produce a thin film on the framework 52.

As discussed in association with FIG. 4, the deposition reactor 40 can include one or more positions P1 and P2 at which the vapor deposition flow mixture can be deposited on the framework 52. Although example positions P1 and P2 are shown as different positions along a vertical direction of the deposition reactor 40, the one or more positions are not so limited. For example, positions P1 and P2 can, additionally or alternatively, include different positions along a lateral dimension or depth dimension of the deposition reactor 40. Each of the one or more positions P1 and P2 can correlate to unique deposition process variations, including, but not limited to, vapor deposition rate, temperature, vapor flow distribution, and vapor concentration. The one or more positions P1 and P2 can be located below the opening 58 or located away from the opening 58. For example, the one or more positions P1 and P2 can be located on a moveable platform including the opening 58 or the hot wall furnace 44.

The framework 52 can be oriented at one or both of positions P1 and P2 to aid in achieving a desired porosity or density of a region or portion of the implant 10. For example, the framework 52 can be rotated 180 degrees to achieve a similar porosity on opposing sides of the implant 10. The framework 52 can be orientated so that a structured region 15 (FIG. 1) of the framework 52 includes smaller pores than a non-structured region.

It should be appreciated that while the framework 52 has been indicated to be carbon in the disclosed example, other materials including carboneous materials, such as graphite, can be additionally or alternatively be used. In addition, other open cell materials, such as high temperature ceramics, can also be used. Also, other layers, beyond the disclosed tantalum layer, can be deposited on the framework 52, such as intermediate layers to provide additional strength. Although the present disclosure has been described with reference to a particular method of manufacture, such as chemical vapor deposition, other methods of manufacture can be used. For example, electrodeposition by fused salt electrolysis can be used to deposit tantalum or another metallic material on a carbon or other carboneous or open cell framework 52.

As such, it will be understood that a framework such as framework 52 to be infiltrated and coated with a biocompatible metal or other biocompatible material can be provided by any number of suitable three-dimensional, porous structures, and these structures can be formed with one or more of a variety of materials including but not limited to polymeric materials which are subsequently pyrolyzed, metals, metal alloys, ceramics. In some instances, a highly porous three-dimensional structure will be fabricated using a selective laser sintering (SLS) or other additive manufacturing-type process such as direct metal laser sintering. In one example, a three-dimensional porous article is produced in layer-wise fashion from a laser-fusible powder, e.g., a polymeric material powder or a single-component metal powder, that is deposited one layer at a time. The powder is fused, remelted or sintered, by the application of laser energy that is directed to portions of the powder layer corresponding to a cross section of the article. After the fusing of the powder in each layer, an additional layer of powder is deposited, and a further fusing step is carried out, with fused portions or lateral layers fusing so as to fuse portions of previous laid layers until a three-dimensional article is complete. In certain embodiments, a laser selectively fuses powdered material by scanning cross-sections generated from a 3-D digital description of the article, e.g., from a CAD file or scan data, on the surface of a powder bed. Net shape and near net shape constructs are infiltrated and coated in some instances.

Complex geometries can be created using such techniques. In some instances, a three-dimensional porous structure will be particularly suited for contacting bone and/or soft tissue, and in this regard, can be useful as a bone substitute and as cell and tissue receptive material, for example, by allowing tissue to grow into the porous structure over time to enhance fixation (i.e., osseointegration) between the structure and surrounding bodily structures. Illustratively, a matrix approximating natural cancellous bone or another bony structure can be fabricated. In this regard, a three-dimensional porous structure, or any region thereof, may be fabricated to virtually any desired density, porosity, pore shape, and pore size (e.g., pore diameter). Such structures therefore can be isotropic or anisotropic prior to being infiltrated and coated with one or more coating materials. When coated with one or more biocompatible metals, any suitable metal may be used including any of those disclosed herein such as tantalum, titanium, a titanium alloy, cobalt chromium, cobalt chromium molybdenum, tantalum, a tantalum alloy, niobium, or alloys of tantalum and niobium with one another or with other metals. Illustratively, a three-dimensional porous structure may be fabricated to have a substantially uniform porosity, density, pore shape and/or void (pore) size throughout, or to comprise at least one of pore shape, pore size, porosity, and/or density being varied within the structure. For example, a three-dimensional porous structure to be infiltrated and coated may have a different pore shape, pore size and/or porosity at different regions, layers, and surfaces of the structure. According to certain embodiments of the present disclosure, regions of a three-dimensional porous structure to be infiltrated and coated may have a porosity as low as 55%, 65%, or 75% or as high as 80%, 85%, or 90%, or within any range defined between any pair of the foregoing values. In some embodiments, a non-porous or essentially non-porous base substrate will provide a foundation upon which a three-dimensional porous structure will be built and fused thereto using a selective laser sintering (SLS) or other additive manufacturing-type process. Such substrates can incorporate one or more of a variety of biocompatible metals such as titanium, a titanium alloy, cobalt chromium, cobalt chromium molybdenum, tantalum, or a tantalum alloy.

As illustrated in FIG. 4, a method 30 for varying the density of one or more implant portions can include vapor depositing a material on a framework defined by a plurality of interconnecting interstitial cells configured to receive bone or other biological tissue ingrowth 32. Vapor depositing the material can, for example, include a CVD process in a manner disclosed in U.S. Pat. No. 5,282,861. The CVD process can use the deposition reactor 40, as described in association with FIG. 3.

The method 30 can include controlling a rate of the vapor deposition of the material on the framework 34. The rate of vapor deposition of the material can be controlled by adjusting one or more deposition process variations, including concentration of the vapor deposition, temperature within the deposition reactor, temperature at the position of the framework, position of the framework, or adjusting vapor flow distribution using a shield. The shield, e.g., 64 of FIG. 3, can direct or re-direct a vapor deposition flow mixture to one or more desired areas of the framework, such that material density of an implant at such areas can be controlled.

The density of the material can be varied across the implant's framework to provide specified varying porosities of a first portion of the porous region, including a first plurality of cells, and a second portion of the porous region, including a second plurality of cells 36. Varying the porosity density can include varying a first central tendency diameter of a plurality of pores, e.g., 24 of FIG. 2, included in the first portion and varying a second central tendency diameter of a plurality of pores included the second portion. The first central tendency diameter can be smaller than the second central tendency diameter.

The method 30 can include varying the first density of the first porous portion or the second density of the second porous portion to substantially replicate a predetermined anisotropic property, such as elasticity, density, strength, or stress tolerance. The predetermined anisotropic property can include an anisotropic property of an anatomical component of a patient, such as the body component the implant 10 is intended to augment or replace. For example, the method 30 can include increasing the density of a portion of the implant, such as the first portion, to substantially replicate or match the stress tolerance of cortical bone. Further, the density of a portion of the implant, such as the second portion, can be decreased to substantially replicate or match the stress tolerance of cancellous bone.

In an example, density of the material can be varied to include a structured region, e.g., 15 of FIG. 1, configured to provide a larger topology variation than any topology variation associated with a surrounding porous region. In an example, the first portion of the porous region is located within the structured region and the second portion of the porous region is located outside of the structured region. In an example, the structured region can include a thread, an orifice, a textured surface, a protrusion, or a recess. The density of the implant can be varied according to a desired stress tolerance distribution of the implant. For example, a first portion of the implant can have an increased density to withstand a greater stress than a second portion, which can include a density less than the first portion. In an example, the vapor deposition flow mixture can be directed with a shield to achieve a material distribution on the framework according to a desired stress tolerance distribution for the implant. Varying the density can include varying the density of opposing sides of the implant's framework.

The method 30 can include positioning the framework within a deposition reactor according to a temperature distribution of the deposition reactor. For example, the framework can be positioned at a position P1 that correlates to a lower temperature than a position P2. A lower temperature can correlate to an increase in central tendency of the pore diameter. The framework can further be re-positioned to position P2 within the deposition reactor, where P2 correlates to a higher temperature and decrease in central tendency of the pore diameter. The positioning and re-positioning of the framework can be done according to desired different porosities of the first portion and the second portion of a porous region.

The above Detailed Description includes references to the accompanying drawings, which form a part of the Detailed Description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above Detailed Description is intended to be illustrative, and not restrictive. For example, the above-described examples (or one or more aspects thereof) may be used in combination with each other. Other embodiments can be used, such as by one of ordinary skill in the art upon reviewing the above Detailed Description. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The Abstract is submitted with the understanding that it will not be used to interpret or limit the scope or meaning of the claims.

What is claimed is:

1. An implant, comprising:
a porous region including a plurality of interconnecting interstitial cells configured to receive bone or biological tissue ingrowth, the porous region comprising a first portion that includes a first plurality of the interconnecting interstitial cells and has a first density, and a second portion that includes a second plurality of the interconnecting interstitial cells and has a second density different than the first density.

2. The implant of claim 1, wherein the first portion includes a structured region having a larger topology variation than any topology variation associated with the second portion, the second portion being located outside of the structured region.

3. The implant of claim 2, wherein the structured region includes a threaded region having one or more threads.

4. The implant of claim 1, wherein the first density is greater than the second density.

5. The implant of claim 1, wherein the first density is greater than the second density by an amount corresponding to a chemical vapor deposition (CVD) process variation.

6. The implant of claim 1, wherein the first density is greater than the second density by at least 5 percent.

7. The implant of claim 1 including ligaments and one or more pores, each pore having a smaller size than surrounding interstitial cells.

8. The implant of claim 1, wherein:
the first portion includes one or more pores having a first central tendency of pore diameter; and
the second portion includes one or more pores having a second central tendency of pore diameter, the second central tendency of pore diameter being larger than the first central tendency of pore diameter.

9. The implant of claim 1 including a framework and a material deposited on the framework in one or more different amounts to provide the first and second densities of the first and second portions, respectively.

10. The implant of claim 1, wherein the first density or the second density is configured to substantially match a predetermined anisotropic property.

11. The implant of claim 1, wherein the second portion is configured to provide greater bone or biological tissue ingrowth than the first portion.

12. A method, comprising:
vapor depositing a material on a porous framework providing a plurality of interconnecting interstitial cells configured to receive bone or other biological tissue ingrowth; and
controlling a rate of the vapor deposition of the material on one or more portions of the framework, including varying a density of the material across the framework to provide a first porous portion, including a first plurality of the interconnecting interstitial cells and having a first density, and a second porous portion, including a second plurality of the interconnecting interstitial cells and having a second density less than the first density.

13. The method of claim 12, wherein varying the density of the material across the framework includes:
forming a first central tendency diameter of a plurality of pores included in the first porous portion; and
forming a second central tendency diameter, which is greater than the first central tendency diameter, of a plurality of pores included in the second porous portion.

14. The method of claim 12, wherein varying the density of the material across the framework includes providing the first porous portion at a structured region, the structured region having a larger topology variation than any topology variation associated with the second porous portion.

15. The method of claim 12, wherein vapor depositing the material on the framework includes positioning the framework within a deposition reactor according to a temperature distribution of the deposition reactor.

16. The method of claim 12, wherein varying the density of the material across the framework includes positioning the framework within a deposition reactor according to a predetermined porosity for the first portion and a predetermined porosity for the second portion.

17. The method of claim 12, wherein varying the density of the material across the framework includes selecting a porosity for the first portion or the second portion according to a predetermined stress tolerance distribution of an implant.

18. The method of claim 12, wherein varying the density of the material across the framework includes directing vapor deposition flow using a shield.

19. The method of claim 12, wherein varying the density of the material across the framework includes altering the rate or a material concentration of the vapor deposition.

20. The method of claim 12, wherein varying the density of the material across the framework includes providing the first and second porous portions, on opposing sides of an implant.

21. An implant, comprising:
a porous region including a plurality of interconnecting interstitial cells configured to receive bone or biological tissue ingrowth,
the porous region comprising a first portion that includes a first plurality of the interconnecting interstitial cells and has a first density, and a second portion that includes a second plurality of the interstitial cells specified to have a second density of the cells; and
wherein the second portion is configured to provide a lower stress tolerance and greater bone or biological tissue ingrowth than the first portion.

22. The implant of claim 21, wherein the first portion is positioned at a predetermined stress concentration region realized during implant insertion, use, or removal.
